(19) United States
(12) Patent Application Publication
(10) Pub. No.: US 2009/0017136 A1
(43) Pub. Date: Jan. 15, 2009

(54) Formulation to prevent and treat fibromyalgia, depression and other muscle and nervous system disorders

(76) Inventor: Wayne Gorsek, Boynton Beach, FL (US)

Correspondence Address: HOFFMAN WASSON & GITLER, P.C;CRYSTAL CENTER 2, SUITE 522, 2461 SOUTH CLARK STREET, ARLINGTON, VA, 22202-3843, US

(21) Appl. No.: 11/822,750

(22) Filed: Jul. 10, 2007

(51) Int. Cl. A61K 31/12; A61K 31/34; A61K 31/40; A61K 31/59; A61K 31/70; A61K 33/00; A61K 33/32; A61P 21/00

(52) U.S. Cl. 424/641; 424/702; 424/722; 514/167; 514/419; 514/474; 514/52; 514/690

(57) ABSTRACT

The formulation provides for the treatment and prevention of fibromyalgia, depression and other muscle and nervous system disorders. The formulation combines Coenzyme Q10, vitamin B1, 5-Hydroxytryptophan, magnesium, Vitamin D, Vitamin C, Selenium and Zinc. Each component has an effect on its own combined with a synergistic effect with other components.

### BACKGROUND OF THE INVENTION

The formulation embodying the present invention promotes energy production to prevent, treat and reverse fibromyalgia, depression and other muscle and nervous system disorders related to oxidation and mitochondrial dysfunction and/or lack of serotonin.

Fibromyalgia is a complex syndrome with no known cause or cure. Its predominant symptom is pain in the fibrous tissues, muscles, tendons, and ligaments, although other symptoms may be experienced.

Research has demonstrated that the axis connecting the three glands primarily responsible for the stress response (hypothalamus, pituitary, adrenals) may be dysfunctional in people with fibromyalgia. Inflammation of the involved structures is generally absent in fibromyalgia.

Of the estimated three to six million people affected by this disorder in the United States, the vast majority are women between 25 and 45 years of age.

### SUMMARY OF THE INVENTION

The formulation provides for the treatment and prevention of fibromyalgia, depression and other muscle and nervous system disorders. The formulation combines Coenzyme Q10, vitamin B1 (Thiamine HCl), 5-Hydroxytryptophan (5-HTP), magnesium (hydroxide), Vitamin D-3 (Cholecalciterol), Vitamin C (ascorbic acid), Selenium (Selenomethionine) and Zinc (L-Methionine). Each component has an effect on its own combined with a synergistic effect with other components.

### DETAILED DESCRIPTION OF THE INVENTION

The composition has the following essential components in the listed ranges:

| | |
|---|---|
| 5-Hydroxytryptophan (5-HTP) | 20 mg-10,000 mg |
| Coenzyme Q10 | 20 mg-10,000 mg |
| Magnesium (hydroxide) | 40 mg-20,000 mg |
| Vitamin B1 (Thiamine HCl) | 10 mg-5,000 mg |
| Vitamin D-3 (Cholecalciterol) | 200 IU-100,000 IU |
| Vitamin C (ascorbic acid) | 25 mg-10,250 mg |
| Selenium (Selenomethionine) | 20 mcg-10,000 mcg |
| Zinc (L-Methionine) | 3 mg-1,500 mg |

Optional ingredients (Kosher gelatin [capsule], cellulose, silicon dioxide, and magnesium stearate).

One embodiment of the invention has the following amounts of each component:

| | | |
|---|---|---|
| 5-Hydroxytryptophan | 200 | mg |
| Coenzyme Q10 | 200 | mg |
| Magnesium (as hydroxide) | 400 | mg |
| Vitamin B1 Thiamine | 100 | mg |
| Vitamin D | 2,000 | IU |
| Vitamin C (as acorbic acid) | 250 | mg |
| Selenium | 200 | mcg |
| Zinc (as L-Opti) | 30 | mg |

5-HTP, or 5-hydroxytryptophan, is a precursor to the neurotransmitter serotonin—which is essential for controlling mood, sleep and pain levels. The body produces 5-HTP from tryptophan, an amino acid found in small amounts in high-protein foods such as chicken, fish, beef and dairy products. It can also be taken in supplemental form, in which case it's extracted from the seeds of the Griffonia simplicifolia plant.

5-HTP is believed to help increase serotonin production in the brain, which may positively influence mood. 5-HTP may also help with other health issues related to serotonin deficiency, including nervousness, body tension and stiffness and head cavity discomfort.

Coenzyme Q10 (CoQ10), or ubiquinone, is a naturally-occurring nutrient found in cell mitochondria, where energy is produced. The heart, with greater energy requirements than other organs, contains significantly higher amounts of CoQ10 than other tissues in the body. Unfortunately, levels of CoQ10 naturally decline with age, which may result in less efficient functioning of the heart and other organs.

Magnesium is an essential mineral to the human body. It is needed for bone, protein, and fatty acid formation, making new cells, activating B vitamins, relaxing muscles, clotting blood, and forming adenosine triphosphate (ATP; the energy the body runs on). The secretion and action of insulin also require magnesium.

Vitamin B1 is a water-soluble vitamin needed to process carbohydrates, fat, and protein. Every cell of the body requires vitamin B1 to form the fuel the body runs on-adenosine triphosphate (ATP). Nerve cells require vitamin B1 in order to function normally.

The fat-soluble vitamin D's most important role is maintaining blood levels of calcium, which it accomplishes by increasing absorption of calcium from food and reducing urinary calcium loss. Both effects keep calcium in the body and therefore spare the calcium that is stored in bones. When necessary, vitamin D transfers calcium from the bone into the bloodstream, which does not benefit bones. Although the overall effect of vitamin D on the bones is complicated, some vitamin D is necessary for healthy bones and teeth.

Vitamin C is important for connective tissue repair. Although beneficial to athletes participating in a variety of sports, vitamin C is especially important to individuals that have connective tissue damage.

Vitamin C is also important as an antioxidant, it may help to reverse some of the oxidative damage that may occur from exercise. This oxidative damage, caused by free radicals, may interfere with the cells' ability to function normally and is believed to play a role in many different health conditions, including the aging process, cancer, and heart disease.

Vitamin C promotes a healthy immune system and may help to prevent the dip in immune function that may occur right after exercise.

Selenium is an essential trace element for humans and other animals.

Selenium is incorporated into molecules of an enzyme called glutathione peroxidase (GPX). This vital enzyme protects red blood cells and cell membranes against undesirable reactions with soluble peroxides.

The dependence of glutathione peroxidase on nutritional selenium clarifies the antioxidant role of this essential micronutrient. Good selenium nutrition is of key importance for antioxidant defense as well as efficient energy metabolism.

Zinc is also needed for the repair of damaged tissue and has protected against stomach ulceration in animal studies.

Zinc salt of the amino acid carnosine exerts significant protection against ulcer formation and promotes the healing of existing ulcers.

I claim:

1. A formulation to treat, and prevent fibromyalgia, depression and muscle and nervous system disorders comprising:
5-Hydroxytryptophan;
Coenzyme Q10;
Magnesium hydroxide;
Vitamin B1;
Vitamin D;
Vitamin C;
Selenium; and
Zinc.

2. The formulation to treat, and prevent fibromyalgia, depression and muscle and nervous system disorders as claimed in claim 1 comprising:

| | |
|---|---|
| 5-Hydroxytryptophan (5-HTP) | 20 mg-10,000 mg |
| Coenzyme Q10 | 20 mg-10,000 mg |
| Magnesium (hydroxide) | 40 mg-20,000 mg |
| Vitamin B1 (Thiamine HCl) | 10 mg-5,000 mg |
| Vitamin D-3 (Cholecalciterol) | 200 IU-100,000 IU |
| Vitamin C (ascorbic acid) | 25 mg-10,250 mg |
| Selenium (Selenomethionine) | 20 mcg-10,000 mcg |
| Zinc (L-Methionine) | 3 mg-1,500 mg. |

3. The composition of claim 1, further comprising

| | | |
|---|---|---|
| 5-Hydroxytryptophan | 200 | mg; |
| Coenzyme Q10 | 200 | mg; |
| Magnesium hydroxide | 400 | mg; |
| Vitamin B1 Thiamine | 100 | mg; |
| Vitamin D | 2,000 | IU; |
| Vitamin C (as acorbic acid) | 250 | mg; |
| Selenium | 200 | mcg; and |
| Zinc | 30 | mg. |
